(19) United States
(12) Patent Application Publication
(10) Pub. No.: US 2009/0216070 A1
(43) Pub. Date: Aug. 27, 2009

(54) APPARATUS AND METHOD OF RELAXATION THERAPY

(76) Inventors: Ronald D. Hunt, Waxhaw, NC (US); David R. Fry, Waxhaw, NC (US)

Correspondence Address: ADAMS INTELLECTUAL PROPERTY LAW, P.A., Suite 2350 Charlotte Plaza, 201 South College Street, CHARLOTTE, NC, 28244, US

(21) Appl. No.: 12/392,550

(22) Filed: Feb. 25, 2009

Related U.S. Application Data
(60) Provisional application No. 61/031,062, filed on Feb. 25, 2008.

(51) Int. Cl. A61M 21/00; A63F 13/00; H04N 5/91

(52) U.S. Cl. 600/27; 463/31; 38/96

(57) ABSTRACT

Apparatus and methods are provided for contemporaneously displaying video images, delivering audio signals and emitting a scent to an individual. A visual sensory portion includes a video screen for displaying the video images to the user. An auditory sensory portion includes a speaker for delivering the audio signals to the user. An olfactory sensory portion includes a renewable scent element impregnated with an aroma for emitting the scent to the user. A remote controller may be provided for controlling the video images displayed to the user and for controlling the audio signals delivered to the user. A disposable shield may be provided for attachment to eyewear worn by the user for shielding the eyes of the user from extraneous visual distractions. The apparatus may be used in relaxation therapy to produce a comprehensive state of relaxation in the patient prior to a medical, dental or psychological procedure.

### CROSS REFERENCE TO RELATED APPLICATION

This application claims the benefit of U.S. Provisional Patent Application No. 61/031,062 filed on Feb. 25, 2008, the disclosure of which is incorporated herein in its entirety.

### FIELD OF THE INVENTION

The present invention relates to apparatus and methods of relaxation therapy, as well as entertainment, recreation (e.g. gaming), sports motivation and training. More particularly, the invention is an apparatus and method for contemporaneously displaying video images, delivering audio signals and emitting a scent to an individual. In a preferred embodiment, the invention is a therapeutic device that incorporates the senses of sight, sound and smell to produce a comprehensive state of relaxation in an individual, for example a patient prior to a medical, dental or psychological procedure.

### BACKGROUND OF THE INVENTION

Relaxation therapy has long been enjoyed by people as a means for relieving stress, reducing anxiety and enhancing the beneficial effects of sleep. It is well known that the senses of sight, sound and smell can each be stimulated to produce a state of relaxation. As a result, tranquil scenes, soothing sounds and pleasant scents are commonly utilized in relaxation therapy. Massage therapy, for example, typically utilizes soothing sounds, such as nature noises (e.g. water, birds, crickets, etc.), to help the recipient achieve an enhanced state of relaxation. Aromatherapy is utilized in certain clinical settings to elicit a desired response, such as calmness or drowsiness. It is also commonplace for relaxation therapy to combine tranquil video images and soothing audio, or to include a pleasant scent along with soothing sounds (e.g. massage therapy), to provide a more comprehensive state of relaxation.

In recent years, the administration of sedation, anesthesia and associated medications has become a principal cost of medical, dental and psychological procedures. At the same time, the benefits of alternative medicine, including reduced cost, are become increasingly recognized and accepted by patients and insurance carriers alike. It is believed that alternative medicine, and in particular relaxation therapy, significantly reduces the amount of anesthesia and medication required to be administered to a patient for a given procedure. In some instances, relaxation therapy can eliminate the need for sedation or anesthesia altogether. The health care community, however, has not yet widely accepted the use of relaxation therapy as a method for supplementing or supplanting sedation, anesthesia and medications in ordinary medical dental and psychological procedures. This is believed to be due at least in part to the lack of proven, convenient and cost-effective apparatus and methods for achieving a comprehensive state of relaxation in a patient.

Thus, it is apparent a need exists for a proven, convenient and cost effective apparatus and method for contemporaneously displaying video images, delivering audio signals and emitting scent to an individual. A more specific need exists for an apparatus and method of relaxation therapy that combines the senses of sight, sound and smell to produce a comprehensive state of relaxation in an individual. In a preferred embodiment, the invention is a therapeutic device that incorporates video images, audio signals and scent to produce a comprehensive state of relaxation in a patient prior to a medical, dental or psychological procedure.

### SUMMARY OF THE INVENTION

In one aspect, the invention is an apparatus for contemporaneously displaying video images, delivering audio signals and emitting scent to an individual. The apparatus includes a visual sensory portion, an auditory sensory portion and an olfactory sensory portion. The visual sensory portion includes a video display system configured for displaying video images to a user. The auditory sensory portion includes an audio delivery system configured for delivering audio signals to the user. The olfactory sensory portion includes a scent emitter system configured for emitting a scent to the user.

In one embodiment, the video display system includes at least one video screen coupled to a video source and is operable to display the video images to the user via the video screen. The audio delivery system includes at least one speaker coupled to an audio source and is operable to deliver the audio signals to the user via the speaker. The scent emitter system includes a scent emitter and is operable to emit the scent to the user via the scent emitter.

In another embodiment, the video display system includes at least one video screen disposed on a frame portion of eyewear worn by the user. The audio delivery system includes at least one speaker disposed on an ear stem portion of the eyewear. The scent emitter system includes a scent emitter disposed adjacent a nose bridge portion of the eyewear. By way of example, and not for purposes of limitation, the eyewear may be conventional virtual reality glasses, or goggles, and the at least one speaker may be a conventional earpiece or earphone disposed on the virtual reality glasses. Furthermore, the scent emitter may be a generally porous, relatively absorbent material impregnated with an aroma.

In yet another embodiment, the apparatus further includes a remote controller for controlling the video images displayed to the user via the video screen and for controlling the audio signals delivered to the user via the speaker. By way of example, and not for purposes of limitation, the remote controller may be a portable audio/video player, such as an IPOD® or MP3 player. The remote controller may be hard-wired to the eyewear, or alternatively, may be in wireless communication with the video screen and the speaker. Alternatively, the frame portion of the eyewear may include a media port for receiving a video source containing the video images to be displayed to the user.

In yet another embodiment, the apparatus further includes a shield configured for attachment to the eyewear worn by the user. The shield is operable for shielding the eyes of the user from extraneous visual distractions. The shield has at least one cavity open to an exterior surface of the shield adjacent the nose bridge portion of the eyewear for receiving a scent element impregnated with an aroma. If desired, the shield may be disposable and the scent element may be renewable with the same or a different aroma. The shield is made of a relatively pliable material and is shaped to conform generally to an outer contour of the eyewear. By way of example and for purposes of limitation, the shield may be made of a generally opaque, closed-cell polyurethane foam material.

In another aspect, the invention is a therapeutic device for providing relaxation therapy to a patient. The device includes eyewear adapted to be worn by the patient, the eyewear having a frame portion, a nose bridge portion and at least one ear stem portion. At least one video screen is disposed on the frame portion of the eyewear and is configured for displaying video images to the patient. At least one speaker is disposed on the ear stem portion of the eyewear and is configured for delivering audio signals to the patient. At least one scent emitter is disposed adjacent the nose bridge portion of the eyewear and is configured for emitting a scent to the patient.

In one embodiment, the therapeutic device further includes a shield configured for attachment to the eyewear worn by the patient. The shield is made of a disposable, relatively pliable, generally opaque material, such as a closed-cell polyurethane foam material, and is shaped to conform generally to an outer contour of the eyewear. The shield has at least one, and preferably a plurality, of cavities adjacent the nose bridge portion of the eyewear. Each cavity is open to an exterior surface of the shield adjacent the nose bridge portion of the eyewear and is configured for receiving a scent element impregnated with an aroma. Preferably, the scent element is made of a relatively absorbent material, such as a conventional cotton ball, impregnated with a liquid aromatherapy oil.

In yet another aspect, the invention is a method for contemporaneously displaying video images, delivering audio signals and emitting a scent to an individual. The method includes providing a video display system having at least one video screen for displaying the video images to the individual via the video screen. The method further includes providing an audio delivery system having at least one speaker for delivering the audio signals to the individual via the speaker. The method further includes providing a scent emitter system having at least one scent emitter for emitting the scent to the individual via the scent emitter.

In still another aspect, the invention is a method of providing relaxation therapy to a patient. The method includes providing a video display system having at least one video screen for displaying video images to the patient. The method further includes providing an audio delivery system having at least one speaker for delivering audio signals to the patient. The method further includes providing a scent emitter system having at least one scent emitter for emitting a scent to the patient. The method further includes contemporaneously displaying the video images to the patient via the video screen, delivering the audio signals to the patient via the speaker, and emitting the scent to the patient via the scent emitter to produce a comprehensive state of relaxation in the patient prior to a medical, dental or psychological procedure. Preferably, the video images, the audio signals and the scent are selected from predetermined sights, sounds and smells that collectively provide a comprehensive state of relaxation to the patient.

### BRIEF DESCRIPTION OF THE DRAWINGS

The invention is best understood when the following detailed description is considered in conjunction with the accompanying drawing figures in which like reference numerals in the different views indicate the same or similar parts.

FIG. 1 is a front perspective view showing a preferred embodiment of an apparatus for contemporaneously displaying video images, delivering audio signals and emitting a scent in accordance with the invention.

FIG. 2 is an enlarged perspective view of the container of the scent emitter system for use with the olfactory sensory portion of the apparatus of FIG. 1 showing the container in an opened configuration.

FIG. 3 is an enlarged perspective view of the container of the scent emitter system of FIG. 2 showing the container in a closed configuration.

FIG. 4 is a front partial perspective and partial plan view illustrating a preferred embodiment of a method for contemporaneously displaying video images, delivering audio signals and emitting a scent in accordance with the invention utilizing the apparatus of FIG. 1.

FIG. 5 is a rear partial perspective and partial plan view illustrating a preferred embodiment of a method of relaxation therapy in accordance with the invention utilizing the apparatus of FIG. 1.

FIG. 6 is a front perspective view showing another preferred embodiment of an apparatus and method for contemporaneously displaying video images, delivering audio signals and emitting a scent in accordance with the invention.

FIG. 7 is an exploded front perspective view showing the various components of the apparatus of FIG. 6 in greater detail.

### DETAILED DESCRIPTION OF PREFERRED EMBODIMENTS OF THE INVENTION

Various preferred embodiments of an apparatus and method for contemporaneously displaying video images, delivering audio signals and emitting a scent to an individual are described in greater detail below and are shown and described in the accompanying drawing figures. While preferred embodiments are presented herein, the scope of the invention is not intended to be limited in any manner to the preferred embodiments shown and described. To the contrary, the invention is intended to be limited only by the scope of the appended claims, as interpreted according to known and accepted principles of patent law. In at least one preferred embodiment, an apparatus constructed in accordance with the invention is useful in a method of providing relaxation therapy. The apparatus is configured to utilize the senses of sight, sound and smell to produce a comprehensive state of relaxation in an individual.

In at least one other preferred embodiment, an apparatus constructed in accordance with the invention is particularly useful in a method of reducing the state of anxiety of a patient prior to a medical, dental or psychological procedure, such as an invasive surgery or hypnosis. The reduced state of anxiety of the patient has the beneficial effect of reducing the amount of anesthesia or medication required to sedate the patient sufficiently to perform the procedure. In at least one other preferred embodiment, an apparatus constructed in accordance with the invention is useful in a method of altering, and in particular, improving the mental state of an individual. For example, the apparatus may be used to stimulate, motivate or invigorate a person to improve his or her performance of an activity. Similarly, the apparatus may be used to increase the confidence of a person or convince a person of his or her abilities. For example, the apparatus may be utilized by an athlete prior to an athletic competition or endeavor to visualize a desired performance or result. Furthermore, an apparatus in accordance with the invention may be used in a method of teaching, tutoring or training an individual.

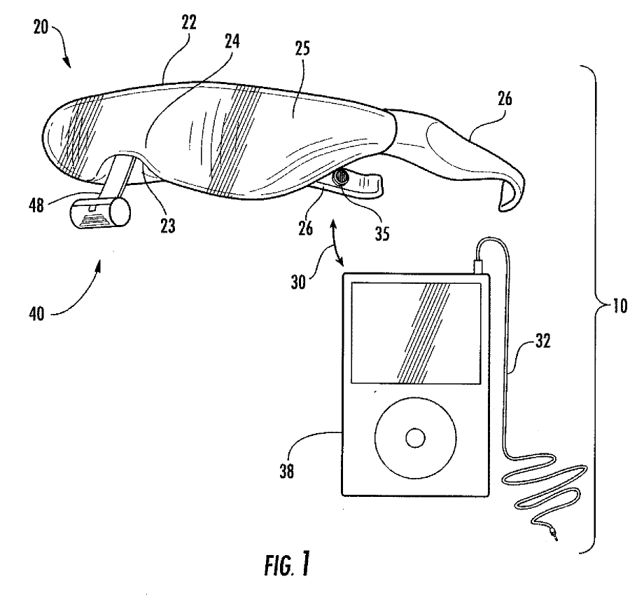

In a preferred embodiment shown in FIG. 1, an apparatus in accordance with the invention, indicated generally at 10, comprises a visual sensory portion 20, an auditory sensory portion 30 and an olfactory sensory portion 40. The visual sensory portion 20, the auditory sensory portion 30 and the olfactory sensory portion 40 combine together in a synergistic manner to contemporaneously display video images, deliver audio signals and emit a scent to an individual. In particular, the visual sensory portion 20 comprises a video display system operable for displaying video images consisting of, for example, tranquil scenes. The auditory sensory portion 30 comprises an audio delivery system operable for delivering audio signals consisting oft for example, soothing sounds. The olfactory sensory portion 40 comprises a scent emitter system operable for emitting a scent, for example, a pleasant aroma of an aromatherapy oil. The contemporaneous combination of tranquil scenes, soothing sounds and pleasant aromas produces a comprehensive state of relaxation utilizing proven, convenient and cost-effective relaxation therapy techniques. As previously mentioned, relaxation therapy in general has been shown to significantly reduce, or even entirely eliminate, the amount of anesthesia or other medication required to sufficiently sedate a patient for a medical, dental or psychological procedure. Accordingly, it is believed that the present invention can significantly reduce the cost of such procedures. Relaxation therapy has also been shown to motivate, stimulate or invigorate a person, and to increase the confidence of, or convince a person of his or her abilities. As such, the synergistic effects of the comprehensive relaxation therapy provided by the present invention can also be utilized to improve a person's performance of an activity, such as an amateur or professional sport.

In the preferred embodiments shown and described herein, the video display system comprises a headset or eyewear 22 adapted to be worn by an individual in the same manner as conventional glasses or goggles. The eyewear 22 is similar in structure and function to conventional virtual reality glasses or goggles commonly utilized in video gaming and other forms of interactive entertainment. Eyewear 22 suitable for use with the invention includes any known portable, personal entertainment device for viewing video images (e.g., movies, videos, etc.) and/or listening to prerecorded audio signals (e.g., music, motivational speeches, audio files, etc.). A first example of eyewear 22 suitable for use with the invention and shown in FIG. 1 is commercially known as ezVision® Video Glasses available from AudioOutfitters, LLC of Murray, Utah. The eyewear 22 comprises a frame portion 25 including a nose bridge portion 24 and a pair of ear stem portions 26 depending rearward, relative to the user, from the nose bridge portion 24. The nose bridge portion 24 has a contoured recess 23 medially disposed between the ear stem portions 26 for comfortably supporting the eyewear 22 on the bridge of the nose of the user. Finally, the video display system, and in particular the eyewear 22, comprises at least one video screen 28 (FIG. 5) for displaying the video images to the user. In a preferred embodiment, the video screen(s) simulate a relatively large (e.g. 50″) virtual video screen for the user. Furthermore, the eyewear 22 is preferably made of an ultra lightweight material, such as plastic, and fits comfortably on the face of the user similar to conventional sunglasses.

The audio delivery system comprises at least one speaker 35 operably coupled to an audio source 38 for generating audio signals (i.e. audible sound). Similarly, the at least one video screen 28 is operably coupled to a video source for generating the video images. As shown, the audio source 38 also serves as the video source for the video screen(s) 28. Any conventional video/audio or audio/video source 38 can be configured for use with the invention. An example of a combination audio and video source particularly well suited for use with the invention is a portable, personal DVD player, such as the ezGear® PL100 ezPlay Screenless DVD Player available from AudioOutfitters, LLC of Murray, Utah that is configured for use with the ezVision Video Glasses. However, an audio/video iPod® player available from Apple Inc. of Cupertino, Calif. is also a suitable video/audio source 38, as well as a conventional MP3 player. Typically, the audio/video source 38 will also include a power source, such as a rechargeable (e.g. lithium) battery and a recharging adapter. The eyewear 22 may be powered by the power source or may be provided with its own lightweight power source, such as a conventional replaceable or rechargeable battery. The audio/video source 38 may be operably coupled to the eyewear 22 by an electrical or optical communications cable 32. Alternatively, the audio/video source 38 may comprise a wireless radio frequency (RF) transmitter and the eyewear 22 provided with a wireless RF receiver for receiving the video images and the audio signals. Typically, the audio/video source 38 is configured for use with digital video and audio files stored on a compact disc (CD), digital video disc (DVD) or compact disc with graphics (CDG) format. However, any suitable video or audio file format and media may be used to provide the video images and audio signals to the eyewear 22. As shown, the at least one speaker 35 is disposed on one or both of the ear stem portions 26 adjacent an ear of the user. The speaker 35 may be external to the eyewear 22, or one or more conventional earpieces may be operably coupled to the audio/video source 38 for insertion into the ears of the user.

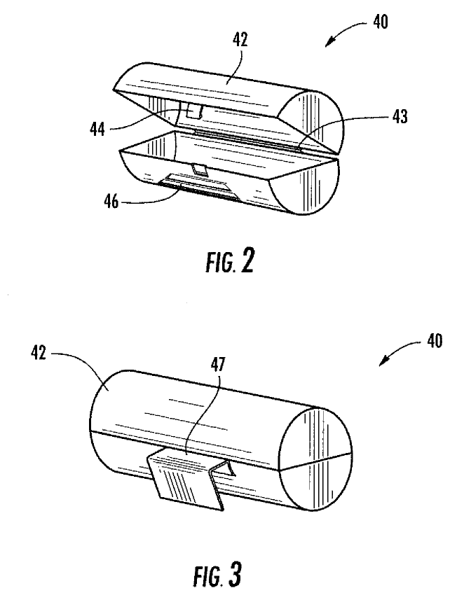

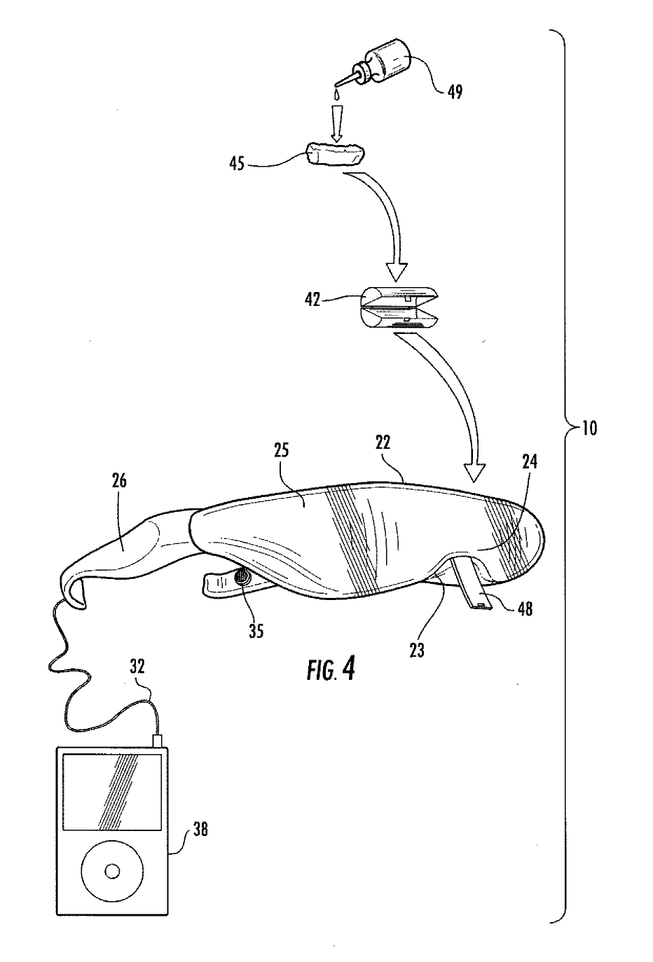

As best shown in FIGS. 2 and 3, the scent emitter system comprises a container, also referred to as the aroma box, 42 for storing a renewable scent element 45. As used herein, the term “renewable” means that the scent element 45 can be removed and replaced with a new or different scent element, or can be removed, the scent renewed, and replaced. The renewable scent element may be any device or component suitable for emitting an aroma to the user. Preferably, the scent element 45 is formed of a relatively absorbent material, such as a conventional cotton ball, capable of being impregnated with an aroma, such as liquid aromatherapy oil. Alternatively, the scent element 45 may be formed of a relatively non-absorbent material that is naturally scented (e.g. eucalyptus leaf, or that has a pre-selected scent applied thereto, for example a coating containing a cinnamon, vanilla or lavender extract. It is anticipated that a variety of different scents will be made available to the user for use with different video images and audio signals. As such, the scent emitted by the scent emitter system will be readily associated with the video images displayed by the video display system and the audio signals delivered by the audio delivery system. In a preferred embodiment, the scent element 45 comprises a plurality of vials 49 each containing a liquid aromatherapy oil, in the manner illustrated in FIG. 5.

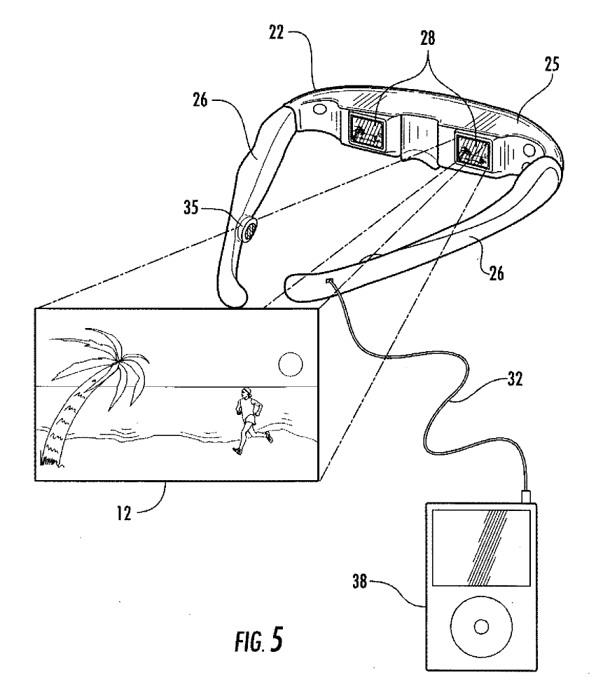

As shown, the container 42 is elongate in the lateral direction (relative to the frame portion 25 of the eyewear 22) so as to extend laterally beyond the bridge of the user's nose. However, the container 42 may have any convenient size or shape, dependent only upon the size and shape of the scent element 45 to be stored therein. The container 42 is configured to be repeatedly opened and closed to insert and subsequently remove the scent element 45 from the container, as desired. In the preferred embodiment shown herein, the container 42 has a laterally extending hinge 43 and a clasp 44 or other closure opposite the hinge for securing the container in the closed configuration (FIG. 3). The container 42 also has at least one, and preferably, a plurality of openings or vents 46 formed therethrough for permitting the scent of the scent element 45 to permeate from the container. The vents 46 may have any suitable size or shape. Preferably, however, the vents 46 preferably extend in a lateral direction across the lower portion of the container 42 so as to be in a proximate location to the nostrils of the nose of the user. The container 42 is also provided with a clip 47 for attaching the container to the nose bridge portion 24 of the eyewear 22. In addition, the clip 47 (or the nose bridge portion 24, as shown in FIG. 1 and FIG. 4) may comprise an extension 48 to position the container 42 at a predetermined distance from the eyewear 22 adjacent the nostrils of the nose of the user.

A preferred embodiment of a method of contemporaneously displaying video images, delivering audio signals and emitting a scent to an individual in accordance with the invention using the apparatus 10 is illustrated in FIG. 4. A preferred embodiment of a method of relaxation therapy in accordance with the invention using the apparatus 10 to produce a comprehensive state of relaxation in a person, for example a patient prior to a medical, dental or psychological procedure, is illustrated in FIG. 5. Suitable video images and audio signals are selected from a plurality of previously produced relaxation therapy audio/video programs (not shown). The audio/video programs may be selected by the user (e.g. patient), or may be recommended by a qualified relaxation therapist or technician. A scent element 45 associated with the video images and/or audio signals is then selected to be utilized with the olfactory sensory portion 40 of the invention. In the preferred embodiment shown in FIG. 4, a relatively absorbent material, such as a conventional cotton ball, is impregnated with an aroma, for example liquid aromatherapy oil, from a predetermined vial 49. The impregnated scent element 45 is then positioned within the opened container 42 of the scent emitter system, and the container is subsequently secured in the closed configuration using the clasp 44. The container 42 is then attached to the nose bridge portion 24 of the eyewear 22 adjacent the nose of the user. Preferably, the extension 48 positions the vents 46 of the container 42 in close proximity to the nostrils of the user's nose. The eyewear 22 is placed on the head of the user in a comfortable and convenient position for permitting the user to view the video images on the at least one video screen 28 and to hear the audio signals through the speaker 35. As previously shown and described, the video images and audio signals are provided to the user by the audio/video source 38. In this manner, the user may view, for example, video images 12 of a person walking or running along a tranquil beach, while listening to sounds of the beach or ocean, at the same time the user is experiencing a scent reminiscent of the beach or an ocean breeze.

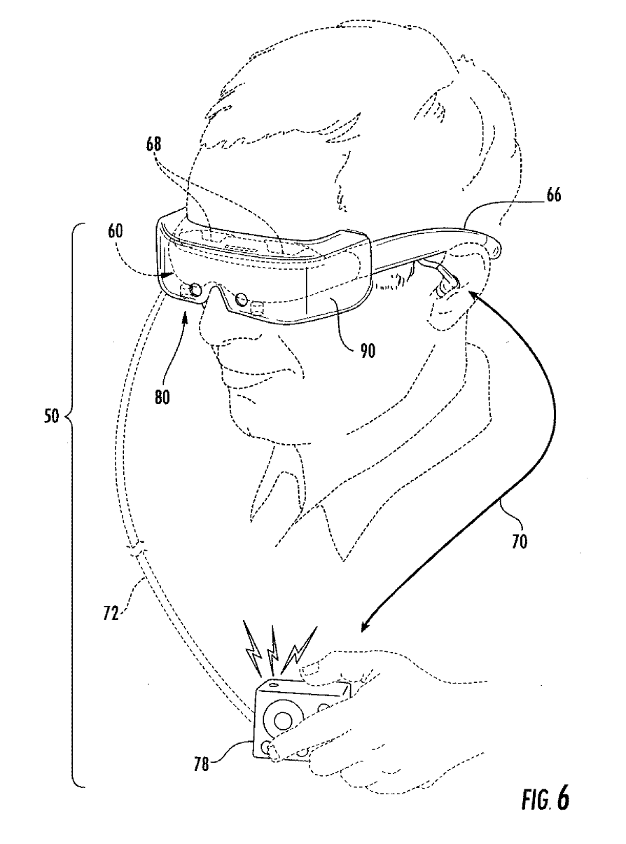

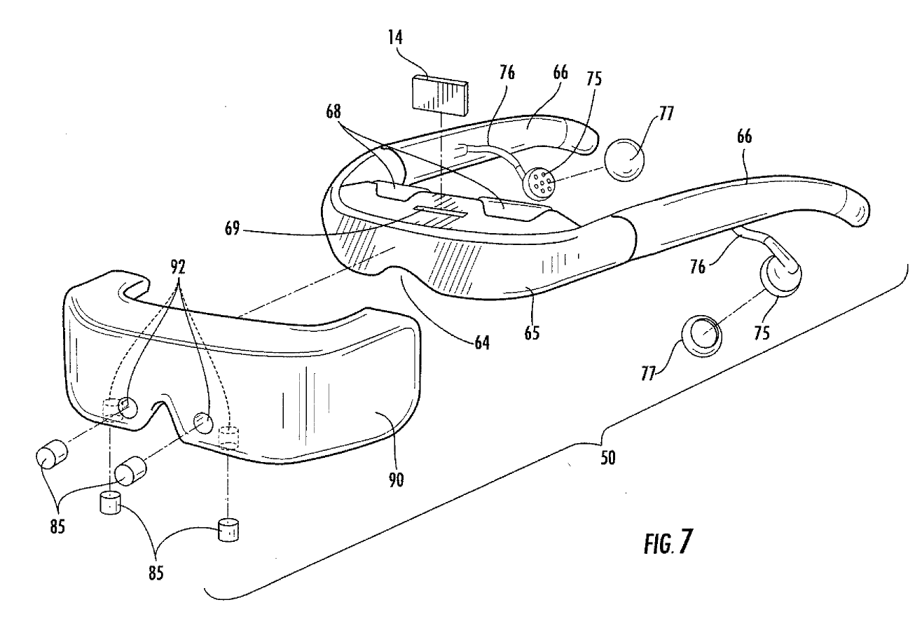

Another preferred embodiment of an apparatus and method for contemporaneously displaying video images, delivering audio signals and emitting a scent in accordance with the invention is shown in FIG. 6. FIG. 7 shows the various components of the apparatus 60 in greater detail. The apparatus, indicated generally at 50, includes a visual sensory portion 60 comprising eyewear 62. An example of eyewear 62 suitable for use with the preferred embodiment of FIGS. 6 and 7 is commercially known as DIGITAL VIDEO GLASSES WITH MP3/MP4 PLAYER AND 80 INCH QVGA available at www.ucables.com as Product Reference R43249. Eyewear 62 comprises a frame portion 65 including a nose bridge portion 64 and a pair of ear stem portions 66 depending rearward from the nose bridge portion 64 essentially as previously described with the exceptions noted herein. Most notably, the frame portion 65 further comprises an elongate slot 69 formed therein above the nose bridge portion 64 of the eyewear 62. The slot 69 defines a media port configured to receive an audio/video source containing the video images to be displayed to the user and/or the audio signals to be delivered to the user. For example, the media port 69 may be configured to receive a conventional memory card 14 containing video files to be displayed to the user via the video screen (not shown) and/or audio files to be delivered to the user via at least one speaker 75. Use of the media port 69 is optional and may replace a remote controller 78 for hands-free use of the apparatus 50. Although not shown, conventional controls, such as a power switch, play, stop/pause, fast forward, fast reverse, etc. may be provided at a convenient location on the eyewear 62, for example on one or both of the ear stem portions 66. Alternatively, the remote controller 78 may be employed for providing the video images and/or audio signals to the user. As previously described, the remote controller 78 may be a portable audio/video player, such as an IPOD® or MP3 player. Furthermore, the remote controller 78 may be hand-held or provided with a clip for attaching to a piece of the user's clothing. The remote controller 78 may be hard-wired to the eyewear via an electrical or optical communications cable 72, or alternatively, may be in wireless (e.g. RF) communication with the video screen 68 and the speaker 75 via a wireless (e.g. RF) receiver disposed within the eyewear 62.

The apparatus 50 further comprises an auditory sensory portion 70 essentially as previously described with the exception that each of the one or more speakers 75 is attached to the ear stem portion 66 of the eyewear 62 by a short length of a flexible electrical cable or cord 76. In this manner, the speaker 75 may be inserted directly into the ear of the user, and thereby function as an earphone or earpiece to deliver enhanced audio signals to the user with minimal extraneous auditory distractions. The auditory sensory portion 70 may optionally comprise a disposable ear bud 77 for temporarily covering the speaker 75. The disposable ear bud 77 is removable and replaceable so as to provide a sanitary earphone or earpiece when the apparatus 50 is intended to be used by multiple individuals, for example different patients in a medical, dental or psychiatric facility. Alternatively, the ear bud 77 may be sanitized after each use, for example using ultraviolet (UV) light. The apparatus further comprises an olfactory sensory portion 80 essentially as previously described with the exception that at least one, and preferably, a plurality of scent elements 85 are disposed adjacent the nose bridge portion 64 of the eyewear 62 within a shield 90 configured for attachment to the eyewear 62 worn by the user. The shield 90 is sized and shaped to conform generally to the outer contour of the eyewear 62, and thus, is operable for shielding the eyes of the user from extraneous visual distractions. As such, the shield 90 is preferably made of a relatively pliable material, for example a generally opaque, closed-cell polyurethane foam material. If desired, formable elements, such as metal wires, may be encased within the material of the shield 90 to permit the shield to conform to the outer contour of the eyewear 62. As shown, the shield 90 has a plurality of cavities 92 open to an exterior surface of the shield for receiving scent elements 85 impregnated with an aroma. Preferably, the shield 90 has at least a pair of opposed cavities 92 that are positioned adjacent the nose bridge portion 64 of the eyewear 62 when the shield 90 is attached to the eyewear. Consequently, the scent elements 85 will be positioned adjacent the nostrils of the user when the eyewear is worn by the user. The shield 90 is preferably disposable (i.e. removable and replaceable) so as to provide a sanitary cover for the eyewear 62 when the apparatus 50 is intended to be used by multiple individuals in the manner and for the purposed previously described. Alternatively, the shield 90 may be sanitized after each use, for example using ultraviolet (UV) light. Furthermore, the scent elements 85 are preferably renewable as previously described.

Various apparatus and methods of contemporaneously displaying video images, delivering audio signals and emitting a scent to an individual according to the invention have been shown and described herein sufficiently to enable one of ordinary skill in the art to make, use and practice the invention. While preferred embodiments exemplary of the best modes of making, using and practicing the invention known at this time have been shown and described, the invention is not intended to be so limited. To the contrary, the invention is intended to be construed broadly and to thereby encompass any and all variations, modifications and equivalents within the scope of the appended claims. Furthermore, the invention is not intended to be limited in any manner to the various uses set forth herein. Instead, an apparatus and method according to the invention may be utilized for any purpose within the reasonable interpretation of the scope of the appended claims.

That which is claimed is:

1. An apparatus for contemporaneously displaying video images, delivering audio signals and emitting a scent to a user, the apparatus comprising:
a visual sensory portion comprising a video display system configured for displaying the video images to the user;
an auditory sensory portion comprising an audio delivery system configured for delivering the audio signals to the user; and
an olfactory sensory portion comprising a scent emitter system configured for emitting the scent to the user.

2. An apparatus according to claim 1, wherein the video display system comprises at least one video screen coupled to a video source and is operable to display the video images to the user via the video screen.

3. An apparatus according to claim 1, wherein the audio delivery system comprises at least one speaker coupled to an audio source and is operable to deliver the audio signals to the user via the speaker.

4. An apparatus according to claim 1, wherein the scent emitter system comprises at least one scent emitter and is operable to emit the scent to the user via the scent emitter.

5. An apparatus according to claim 1, wherein the video display system comprises at least one video screen disposed on a frame portion of eyewear worn by the user, wherein the audio delivery system comprises at least one speaker disposed on an ear stem portion of the eyewear, and wherein the scent emitter system comprises a scent emitter disposed adjacent a nose bridge portion of the eyewear.

6. An apparatus according to claim 5, further comprising a remote controller for controlling the video images displayed to the user via the video screen and for controlling the audio signals delivered to the user via the speaker.

7. An apparatus according to claim 6, wherein the controller is a portable audio/video player.

8. An apparatus according to claim 7, wherein the controller is in wireless communication with the video screen and the speaker.

9. An apparatus according to claim 5, further comprising a shield configured for attachment to the eyewear worn by the user, the shield being operable for shielding the eyes of the user from extraneous visual distractions.

10. An apparatus according to claim 9, wherein the shield has at least one cavity open to an exterior surface of the shield for receiving a scent element impregnated with an aroma.

11. An apparatus according to claim 10, wherein the shield comprises a plurality of cavities open to an exterior surface of the shield adjacent the nose bridge portion of the eyewear, each cavity receiving a scent element impregnated with an aroma.

12. An apparatus according to claim 10, wherein the shield is disposable and wherein the scent element is renewable.

13. An apparatus according to claim 9, wherein the shield is made of a relatively pliable material and is shaped to conform generally to an outer contour of the eyewear.

14. An apparatus according to claim 13, wherein the shield is made of a generally opaque, closed-cell polyurethane foam material.

15. An apparatus according to claim 5, further comprising a disposable ear bud for temporarily covering the at least one speaker.

16. An apparatus according to claim 5, wherein the frame portion of the eyewear has a slot formed therein that defines a media port configured to receive an audio/video source.

17. A therapeutic device for providing relaxation therapy to a patient, the device comprising:
eyewear to be worn by the patient, the eyewear comprising a frame portion, a nose bridge portion and at least one ear stem portion;
at least one video screen disposed on the frame portion of the eyewear, the video screen configured for displaying video images to the patient;
at least one speaker disposed on the ear stem portion of the eyewear configured for delivering audio signals to the patient; and
at least one scent emitter disposed adjacent the nose bridge portion of the eyewear configured for emitting a scent to the patient.

18. A therapeutic device according to claim 17, further comprising a shield configured for attachment to the eyewear worn by the patient to shield the patient from extraneous visual distractions, the shield being made of a disposable, relatively pliable material that is shaped to conform generally to an outer contour of the eyewear, the shield having a plurality of cavities adjacent the nose bridge portion of the eyewear, each cavity being open to an exterior surface of the shield adjacent the nose bridge portion of the eyewear and configured for receiving a scent element impregnated with an aroma.

19. A method for contemporaneously displaying video images, delivering audio signals and emitting a scent to an individual, the method comprising:
providing a video display system comprising at least one video screen for displaying the video images to the individual via the video screen;
providing an audio delivery system comprising at least one speaker for delivering the audio signals to the individual via the speaker; and
providing a scent emitter system comprising at least one scent emitter for emitting the scent to the individual via the scent emitter.

20. A method of providing relaxation therapy to a patient, the method comprising:
providing a video display system comprising at least one video screen for displaying video images to the patient;
providing an audio delivery system comprising at least one speaker for delivering audio signals to the patient;
providing a scent emitter system comprising at least one scent emitter for emitting a scent to the patient; and
contemporaneously displaying the video images to the patient via the video screen, delivering the audio signals to the patient via the speaker, and emitting the scent to the patient via the scent emitter to produce a comprehensive state of relaxation in the patient prior to a medical, dental or psychological procedure.
